(19) United States
(12) Patent Application Publication
(10) Pub. No.: US 2015/0250446 A1
(43) Pub. Date: Sep. 10, 2015

(54) ULTRASOUND DIAGNOSTIC APPARATUS, IMAGE PROCESSING APPARATUS, AND IMAGE PROCESSING METHOD

(71) Applicants: KABUSHIKI KAISHA TOSHIBA, Minato-ku (JP); Toshiba Medical Systems Corporation, Otawara-shi (JP)

(72) Inventor: Yuko KANAYAMA, Nasushiobara (JP)

(73) Assignees: KABUSHIKI KAISHA TOSHIBA, Minato-ku (JP); Toshiba Medical Systems Corporation, Otawara-shi (JP)

(21) Appl. No.: 14/719,626

(22) Filed: May 22, 2015

Related U.S. Application Data
(63) Continuation of application No. PCT/JP2013/081486, filed on Nov. 22, 2013.

(30) Foreign Application Priority Data
Nov. 22, 2012 (JP) 2012-256645
Nov. 21, 2013 (JP) 2013-241378

(51) Int. Cl. A61B 8/06; A61B 8/08; A61B 8/00

(52) U.S. Cl. CPC A61B 8/06; A61B 8/463; A61B 8/485; A61B 8/481; A61B 8/488; A61B 8/5223; A61B 8/4245

(57) ABSTRACT

An image generator which generates data of a plurality of images based on an output from the transmitter/receiver, a blood flow image generator which generates data of a plurality of blood flow images based on an output from the transmitter/receiver, a similarity calculator which calculates similarities between the plurality of images, a specifying processor which specifies at least two images exhibiting a low similarity from the plurality of images based on the similarities, an image selector which selects at least two blood flow images respectively corresponding to scanning times of the specified at least two images from the plurality of blood flow images and a display which displays the selected at least two blood flow images.

### CROSS-REFERENCE TO RELATED APPLICATIONS

This application is a Continuation Application of PCT Application NO. PCT/JP2013/081486, filed Nov. 22, 2013 and based upon and claims the benefit of priority from the Japanese Patent Application NO. 2012-256645, filed Nov. 22, 2012; and No. 2013-241378, filed Nov. 21, 2013, the entire contents of all of which are incorporated herein by reference.

### FIELD

Embodiments described herein relate generally to an ultrasonic diagnostic apparatus, image processing apparatus, and image processing method which visualize the internal state of an subject by transmitting and receiving ultrasonic signals to and from an subject.

### BACKGROUND

Ultrasonic diagnosis enables an observation of how the heart beats or the fetus moves in real time, by simply bringing an ultrasonic probe into contact with the body surface. This technique is highly safe, and hence allows repetitive examination. Furthermore, a system according to ultrasonic diagnosis is smaller in size than other diagnostic apparatuses such as an X-ray diagnostic apparatus, X-ray CT (Computed Tomography) apparatus, and MRI (Magnetic Resonance Imaging) apparatus and can be moved to the bedside to be easily and conveniently used for examination. In addition, ultrasonic diagnosis is free from the influences of exposure using X-rays and the like, and hence can be used in obstetric treatment, treatment at home, and the like.

Recently, with a dramatic improvement in the spatial resolution of an ultrasonic diagnostic apparatus, ultrasonic diagnosis has been increasingly applied to very small regions on the body surface such as the four limbs, fingers, and joints. In addition, there has also been an improvement in sensitivity in a technique of visualizing the flow of blood in the Doppler mode. This makes it possible to observe even weak blood flows. For this reason, ultrasonic diagnosis has become widely used in the rheumatoid arthritis field. When performing rheumatoid arthritis diagnosis, the examiner observes the degree of swelling in a joint mainly in the B mode and observes the degree of an inflammatory blood flow in the Doppler mode. There has also been proposed an evaluation method of scoring the degrees of the respective symptoms.

Arthritis often exhibits different symptoms at different positions even in one joint.

It is therefore necessary to save the image data of a portion where the severest inflammation seems to have occurred, upon observing the overall joint, and perform diagnosis based on the image data, instead of performing diagnosis upon seeing only one slice. In addition, a blood flow sometimes looks different depending on pulsation, and hence it is preferable to select image data corresponding to a phase in which blood flows in a larger amount and use it for diagnosis. As described above, the examiner is required to select image data suitable for diagnosis from many image data obtained by ultrasonic scanning when observing one joint. In addition, the examiner generally observes a plurality of joints per patient. When performing actual examination, therefore, the examiner often performs the following operations: performing scanning while moving a probe in a given region; freezing an image at a given point; reviewing images based on image data temporarily saved in a memory while operating a trackball or the like; selecting an image capturing a blood flow most appropriately; and saving image data concerning the image. This series of procedures can be heavy burden on the examiner.

Note that a similar problem can arise in observation of regions of a subject, other than joints, using an ultrasonic diagnostic apparatus.

### BRIEF DESCRIPTION OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing the arrangement of the main part of an ultrasonic diagnostic apparatus according to the first embodiment.

FIG. 2 is a flowchart showing the operation of the ultrasonic diagnostic apparatus according to this embodiment.

FIG. 3 is a view for explaining a technique of extracting the contour of a joint cavity in this embodiment.

FIG. 4 is a graph showing an example of a time-area curve in this embodiment.

FIG. 5 is a graph plotting image similarities (mean square errors) in this embodiment.

FIG. 6 is a view showing display examples of candidate images in this embodiment.

FIG. 7 is a flowchart showing the operation of an ultrasonic diagnostic apparatus according to the second embodiment.

FIG. 8 is a graph showing an example of a time-area curve in this embodiment.

FIG. 9 is a view showing an example of an ultrasonic image in which motion artifacts appear in this embodiment.

FIG. 10 is a graph for explaining a technique of image data selection in this embodiment.

FIG. 11 is a view for explaining an effect in this embodiment.

FIG. 12 is a view for explaining an effect in this embodiment.

FIG. 13 is a block diagram showing the arrangement of the main part of an ultrasonic diagnostic apparatus according to the third embodiment.

FIG. 14 is a flowchart showing the operation of the ultrasonic diagnostic apparatus according to the third embodiment.

FIG. 15 is a view for explaining a technique of selecting image data according to this embodiment.

FIG. 16 is a view for explaining a technique of selecting image data according to this embodiment.

FIG. 17 is a block diagram showing the arrangement of the main part of an image processing apparatus according to the fourth embodiment.

FIG. 18 is a flowchart showing the operation of the image processing apparatus according to this embodiment.

FIG. 19 is a flowchart showing the operation of an ultrasonic diagnostic apparatus according to the fifth embodiment.

FIG. 20 is a flowchart showing the operation of an ultrasonic diagnostic apparatus according to the sixth embodiment.

### DETAILED DESCRIPTION

In general, according to one embodiment, an ultrasonic diagnostic apparatus includes a transmitter/receiver which repeats ultrasonic transmission/reception with respect to a subject, an image generator which generates data of a plurality of images based on an output from the transmitter/receiver, a blood flow image generator which generates data of a plurality of blood flow images based on an output from the transmitter/receiver, a similarity calculator which calculates similarities between the plurality of images, a specifying processor which specifies at least two images exhibiting a low similarity from the plurality of images based on the similarities, an image selector which selects at least two blood flow images respectively corresponding to scanning times of the specified at least two images from the plurality of blood flow images and a display which displays the selected at least two blood flow images.

Several embodiments will be described with reference to the accompanying drawings.

The first, second, and third embodiments disclose ultrasonic diagnostic apparatuses. The fourth, fifth, and sixth embodiments disclose image processing apparatuses. The same reference numerals in each embodiment denote the same constituent elements, and a repetitive description will be omitted.

### First Embodiment

The first embodiment will be described first.

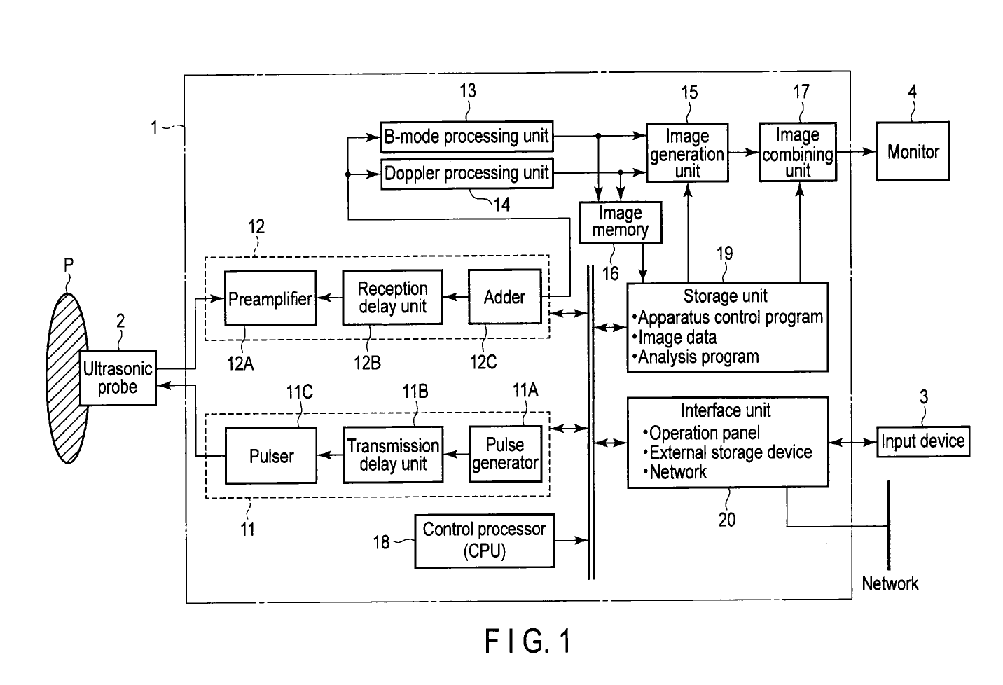

FIG. 1 is a block diagram showing the arrangement of the main part of an ultrasonic diagnostic apparatus according to this embodiment. As shown in FIG. 1, the ultrasonic diagnostic apparatus includes an apparatus main body 1, an ultrasonic probe 2, an input device 3, and a monitor 4.

The apparatus main body 1 includes an ultrasonic transmission unit (an ultrasonic transmitter) 11, an ultrasonic reception unit (an ultrasonic receiver) 12, a B-mode processing unit (a B-mode processor) 13, a Doppler processing unit (a Doppler processor) 14, an image generation unit (an image generator) 15, an image memory 16, an image combining unit (an image combiner) 17, a control processor 18, a storage unit (a storage) 19, and an interface unit (an interface) 20. The ultrasonic transmission unit 11, the ultrasonic reception unit 12, and the like incorporated in the apparatus main body 1 are sometimes implemented by hardware such as integrated circuits and other times by software programs in the form of software modules.

The function of each constituent element will be described below.

The ultrasonic probe 2 has one ultrasonic transducer array corresponding to two-dimensional scanning or a two-dimensional array of ultrasonic transducers corresponding to three-dimensional scanning. The ultrasonic probe 2 includes a plurality of piezoelectric transducers which generate ultrasonic waves based on driving signals from the ultrasonic transmission unit 11 and convert reflected waves from a subject into electrical signals, a matching layer provided for the piezoelectric transducers, and a backing member which prevents ultrasonic waves from propagating backward from the piezoelectric transducers. When the ultrasonic probe 2 transmits ultrasonic waves to a subject P, the transmitted ultrasonic waves are sequentially reflected by a discontinuity surface of acoustic impedance of internal body tissue of the subject P, and are received as an echo signal by the ultrasonic probe 2. The amplitude of this echo signal depends on an acoustic impedance difference on the discontinuity surface by which the echo signal is reflected. The echo signal produced when a transmitted ultrasonic pulse is reflected by the surface of a moving blood flow, cardiac wall, or the like is subjected to a frequency shift depending on the velocity component of the moving body in the ultrasonic transmission direction due to the Doppler effect.

The input device 3 is connected to the apparatus main body 1 and includes various types of switches, buttons, a trackball, a mouse, and a keyboard which are used to input, to the apparatus main body 1, various types of instructions, conditions, an instruction to set an ROI (Region of Interest), various types of image quality condition setting instructions, and the like from an operator.

The monitor 4 displays morphological information and blood flow image in the living body as images based on the video signals supplied from the apparatus main body 1.

The ultrasonic transmission unit 11 includes a pulse generator 11A, a transmission delay unit 11B, and a pulser 11C. The pulser 11C repeatedly generates rate pulses for the formation of transmission ultrasonic waves at a predetermined rate frequency fr Hz (period: 1/fr sec). The transmission delay unit 11B gives each rate pulse a delay time necessary to focus an ultrasonic wave into a beam and determine transmission directivity for each channel. The storage unit 19 stores transmission directions or delay times for deciding transmission directions. The transmission delay unit 11B refers to the delay times stored in the storage unit 19 at the time of transmission. The pulser 11C applies a driving pulse to the ultrasonic probe 2 at the timing based on this rate pulse having passed through the transmission delay unit 11B.

The ultrasonic reception unit 12 includes a preamplifier 12A, an A/D converter (not shown), a reception delay unit 12B, and an adder 12C. The preamplifier 12A amplifies an echo signal received via the ultrasonic probe 2 for each channel. The reception delay unit 12B gives the echo signals amplified by the preamplifier 12A delay times necessary to determine reception directivities. The reception delay unit 12B decides a reception direction or a delay time for deciding a reception direction by referring to the storage unit 19 in the same manner as at the time of the transmission of ultrasonic waves. The adder 12C performs addition processing for the signals having passed through the reception delay unit 12B. With this addition, a reflection component from a direction corresponding to the reception directivity of the echo signal is enhanced to form a composite beam for ultrasonic transmission/reception in accordance with reception directivity and transmission directivity.

As described above, the ultrasonic transmission unit 11 and the ultrasonic reception unit 12 function as a transmission/reception unit which transmits an ultrasonic signal to the subject P and receives an ultrasonic signal (echo signal) reflected by the inside of the subject P.

The B-mode processing unit 13 performs various types of processing such as logarithmic amplification and envelope detection processing for the echo signal received from the ultrasonic reception unit 12 to generate B-mode image data whose signal intensity is expressed by a brightness level. The B-mode processing unit 13 transmits the generated B-mode image data to the image generation unit 15. A B-mode image is a morphological image representing the internal form of a subject.

The Doppler processing unit 14 frequency-analyzes velocity information from the echo signal received from the ultrasonic reception unit 12 to extract a blood flow, tissue, and contrast medium echo component by the Doppler effect, and obtains spatial distributions of average velocities, variances, powers, and the like, i.e., a blood flow image. The Doppler processing unit 14 transmits the obtained blood flow image to the image generation unit 15.

The image generation unit 15 generates B-mode image data as a display image by converting the B-mode image data supplied from the B-mode processing unit 13 into a scanning line signal string in a general video format typified by a TV format. The image generation unit 15 further generates Doppler image data expressing a position at which a blood flow motion is observed by a color pixel with a hue corresponding to an average velocity, variance, or power, based on the blood flow image supplied from the Doppler processing unit 14. The image generation unit 15 incorporates a storage memory which stores B-mode image data and Doppler image data. The operator can retrieve images recorded during examination after, for example, diagnosis.

As described above, the B-mode processing unit 13 and the image generation unit 15 function as a tomographic image generation unit which generates B-mode image data (two-dimensional or three-dimensional morphological image data). The Doppler processing unit 14 and the image generation unit 15 also function as a blood flow image generation unit which generates Doppler image data (blood flow image data) representing the motion state of a blood flow on a slice concerning B-mode image data.

The image memory 16 includes a storage memory which stores the image data generated by the image generation unit 15. For example, the operator can retrieve this image data after diagnosis, and can reproduce the data as a still image or a moving image by using a plurality of frames. The image memory 16 also stores an image brightness signal having passed through the ultrasonic reception unit 12, other raw data, image data acquired via a network, and the like, as needed.

The image combining unit 17 generates display data by combining and superimposing the Doppler image data generated by the image generation unit 15 on the B-mode image data generated by the image generation unit 15. The image combining unit 17 outputs the generated display data to the monitor 4.

The monitor 4 displays an ultrasonic image (B-mode image+Doppler image) based on the display data input from the image combining unit 17. With this operation, the monitor 4 displays the image obtained by color mapping of average velocities, variances, powers, and the like of the moving body on a slice of the subject P represented by brightness.

The storage unit 19 stores a data group including control programs for executing a scan sequence, image generation, and display processing, diagnosis information (a patient ID, findings by a doctor, and the like), and transmission/reception conditions. The storage unit 19 is also used to archive image data in the image memory 16, as needed. It is possible to transfer data stored in the storage unit 19 to an external peripheral device via the interface unit 20.

The control processor 18 is mainly constituted by a CPU (Central Processing Unit) and memories such as a ROM (Read Only Memory) and a RAM (Random Access Memory), and functions as a control unit which controls the operation of the apparatus main body 1. The control processor 18 reads out control programs for executing image generation, display, and the like from the storage unit 19, and executes computation, control, and the like concerning various types of processing.

The interface unit 20 is an interface concerning the input device 3, a network such as a LAN (Local Area Network), and an external storage device (not shown). It is also possible to transfer the image data, analysis result, and the like obtained by the ultrasonic diagnostic apparatus to other apparatuses via the interface unit 20 and a network.

The main operation of the ultrasonic diagnostic apparatus according to this embodiment will be described next.

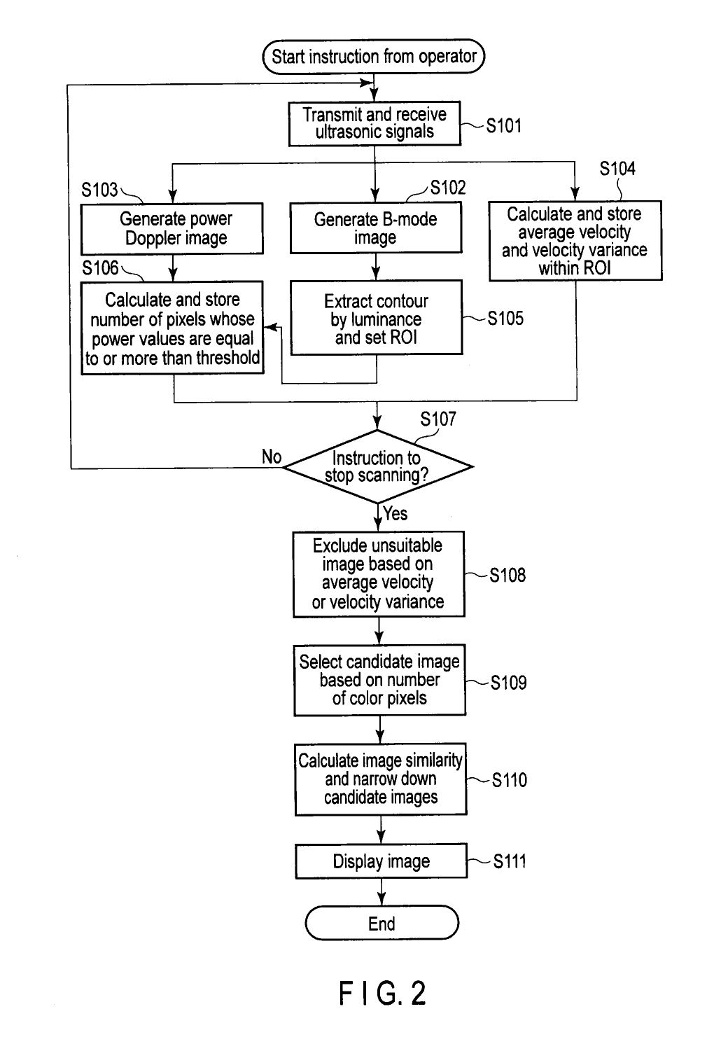

FIG. 2 is a flowchart showing the operation of the ultrasonic diagnostic apparatus. Of the operations shown in this flowchart, the operations in steps S105, S106, and S108 to S110 are implemented by making the control processor 18 execute the analysis program stored in the storage unit 19.

When the operator inputs an instruction to start ultrasonic diagnosis via the input device 3, the control processor 18 instructs the ultrasonic transmission unit 11 and the ultrasonic reception unit 12 to start transmission/reception of ultrasonic waves (step S101). At this time, the ultrasonic transmission unit 11 outputs a transmission signal to the ultrasonic probe 2 in accordance with predetermined settings. Upon receiving this signal, the ultrasonic probe 2 generates an ultrasonic signal into the subject P. In addition, the ultrasonic probe 2 detects the ultrasonic signal (echo signal) returning from the inside of the subject upon reflection and scattering. The ultrasonic reception unit 12 performs reception processing of this echo signal. Assume that in this embodiment, ultrasonic signals to be transmitted and received include a transmission/reception set for the generation of B-mode image data and a transmission/reception set for the generation of Doppler image data, and they are alternately transmitted and received. A signal for the generation of Doppler image data is obtained by consecutively performing transmission/reception a plurality of times on the same scanning line, and velocity information at each position on the scanning line can be obtained by calculating the correlations between a plurality of reception signals.

The B-mode processing unit 13 processes the reception signal for the generation of B-mode image data output from the ultrasonic reception unit 12 in the above manner, and the image generation unit 15 generates grayscale B-mode image data (step S102).

On the other hand, the Doppler processing unit 14 processes the reception signal for the generation of Doppler image data output from the ultrasonic reception unit 12 in the above manner, and the image generation unit 15 generates color scale Doppler image data (step S103). The image generation unit 15 stores the B-mode image data and the Doppler image data generated in steps S102 and S103 in the storage unit 19 in a form that enables the discrimination of phases of image generation. Note that in this embodiment, the Doppler image data generated in step S103 is power Doppler image data expressing the power of a blood flow in color. Note however that the Doppler image data generated in step S103 may be color Doppler image data expressing the velocity of a blood flow in color.

In one of steps S102 and S103, the Doppler processing unit 14 separately processes the reception signal for the generation of Doppler image data to calculate information concerning velocities and the variance of velocities in the first region of interest designated in advance (step S104). The first region of interest is, for example, a color ROI which determines a range in which Doppler image data is generated and displayed on B-mode image data.

The processing in step S104 will be described in detail. When generating a blood flow image in step S103, the Doppler processing unit 14 applies a wall filter (or MTI filter) for cutting low-velocity signals to a reception signal to exclude signals from tissues other than a blood flow. On the other hand, the Doppler processing unit 14 performs correlation computation from a plurality of reception signals obtained on the same scanning line without applying any filter to them in step S104, thereby calculating velocities at the respective points and a variance. This makes it possible to obtain absolute velocity values also considering the motions of tissues other than the blood flow due to body motion, hand movement of the examiner, and the like at the respective points. The Doppler processing unit 14 calculates the average value of velocities, the average value of variances, and the variance value of velocities (or other values as long as they are based on velocity information or variance information) in the entire first region of interest based on the obtained information. Assume that this embodiment uses an average velocity value as an index of body motion or hand movement. The control processor 18 therefore stores the average velocity values calculated in step S104 in association with the Doppler image data generated in step S103 and stored in the storage unit 19.

The control processor 18 then sets segmentation and a region of interest (second region of interest) based on the B-mode image data obtained in step S102 (step S105). More specifically, the control processor 18 extracts the contour of the joint cavity depicted in the B-mode image data, and sets the extracted contour as the second region of interest. The joint cavity is a low-brightness region existing on a bone surface depicted with high brightness. For the extraction of a contour, for example, it is possible to use a technique like that disclosed in Jpn. Pat. Appin. KOKAI Publication No. 2007-190172. In this embodiment, first of all, the operator selects one point contained in a region to be extracted from the B-mode image data by operating the input device 3. The control processor 18 then extracts a region whose brightness around the point selected by the control processor 18 is equal to or less than a threshold designated in advance.

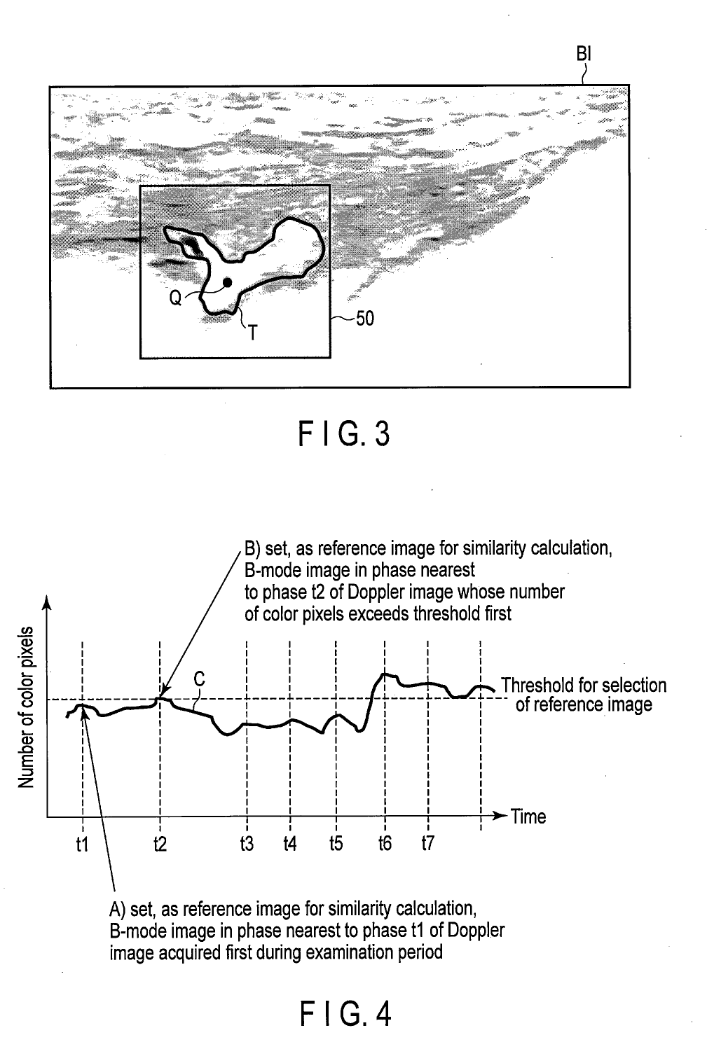

If, for example, the operator designates a point Q as a reference point in B-mode image data BI like that shown in FIG. 3, the control processor 18 extracts a region like a contour T by analyzing the brightness of pixels around the point Q as a starting point. Note that the rectangular frame depicted on the B-mode image data BI in FIG. 3 is a color ROI 50 indicating a range in which Doppler image data is generated and displayed.

In this case, in order to stably perform boundary extraction, the control processor 18 may perform the above extraction processing after performing smoothing processing for B-mode image data. In addition, a region of interest is not always completely surrounded by a high-brightness region. In such a case, the control processor 18 may additionally interpolate a region in which no boundary is detected from a detected partial high-brightness boundary. In addition, it is possible to omit the reference point setting processing performed by the operator. In this case, the control processor 18 may randomly set a plurality of points at which brightness are equal to or less than a predetermined brightness, and may perform boundary extraction by analyzing pixel brightness around the set points. Of the plurality of extracted regions, regions equal to or smaller than a predetermined size are excluded. In addition, in order to exclude the bone surface or a deeper region, a region in contact with the lower end of a screen is excluded.

Of the remaining regions, a region at the deepest level is set as the second region of interest. This makes it possible to set, as the second region of interest, a region, of the low-brightness regions at levels shallower than the bone surface, which is located at the deepest level, i.e., a joint cavity region.

After setting the second region of interest, the control processor 18 calculates the number of color pixels of a Doppler signal contained in the second region of interest set in step S105 in the image data as a parameter representing a characteristic of the Doppler image data generated in step S103 (step S106). More specifically, the control processor 18 calculates the total number of color pixels having power values equal to or more than a preset threshold and contained in the set second region of interest in the Doppler image data generated in step S103. The control processor 18 stores the calculated number of color pixels in association with the Doppler image data generated in step S103 and stored in the storage unit 19.

After steps S104 to S106, the control processor 18 determines whether the operator has input an instruction to stop scanning (step S107). If the operator has not input any instruction (NO in step S107), the process returns to step S101 to repeat steps S101 to S106.

When the operator inputs an instruction to stop scanning by operating the input device 3 (YES in step S107), the control processor 18 executes processing for selecting image data suitable for diagnosis from the plurality of B-mode image data and the plurality of Doppler image data sequentially stored in repeatedly executed steps S102 and S103 (steps S108 to S110).

First of all, the control processor 18 excludes Doppler image data larger in average velocity value in the first region of interest, calculated in step S104, than a predetermined threshold, together with B-mode image data in the same phase, as images having large motion artifacts and regarded as unsuitable as diagnostic images (step S108). Note that it is also possible to exclude image data in step S108 by using, for example, the technique disclosed in Jpn. Pat. Appin. KOKAI Publication No. 9-75344, that is, computing, for each of a plurality of Doppler image data, the ratio of the number of effective pixels exhibiting velocities other than 0 to the all number of pixels constituting one frame and excluding any Doppler image data exhibiting the ratio falling outside the effective range and corresponding B-mode image data. It is also possible to execute step S108 by using a value concerning a velocity variance. In this case, in step S104, the control processor 18 stores a value such as an average variance value calculated by the Doppler processing unit 14 in association with the Doppler image data generated in step S103 and stored in the storage unit 19. In step S108, the control processor 18 excludes, from the candidates, any Doppler image data whose value concerning a variance stored in the storage unit 19 is larger than a predetermined threshold and B-mode image data in the same phase as an image containing a large motion artifact and regarded as unsuitable as a diagnostic image.

The control processor 18 then generates a time-area curve C like that shown in FIG. 4 by plotting the numbers of color pixels calculated in step S106, i.e., the numbers of blood flow pixels, in chronological order (in the order of the phases of image data concerning the respective sets) for all the remaining Doppler image data. The control processor 18 selects candidate image data based on the time-area curve C (step S109). More specifically, the control processor 18 extracts all points on the time-area curve C at which the numbers of color pixels become maximal. For example, in the case shown in FIG. 4, the control processor 18 extracts eight points at t1 to t8. B-mode image data and Doppler image data corresponding to the extracted points are candidate image data.

Subsequently, the control processor 18 calculates image similarities and narrows down candidate image data based on the B-mode image data corresponding to the respective points extracted in step S109 (step S110). In this case, an image similarity is the index obtained by quantifying the degree of similarity between each combination of B-mode image data and Doppler image data corresponding to each point extracted in step S109 and another combination of B-mode image data and Doppler image data. It is possible to use, as an image similarity, the mean square error obtained by, for example, calculating the square root of the arithmetic mean of the square values of the differences between corresponding pixels contained in two image data as comparison targets. In this case, in consideration of the displacement (shift) of B-mode image data, pattern matching may be applied to two image data to adjust pixels, the differences between which should be calculated.

The following is a specific processing procedure.

First of all, the control processor 18 calculates the image similarity (mean square error) between one of the B-mode image data corresponding to the respective points extracted in step S109 and another B-mode image data corresponding to each point extracted in step S109. The control processor 18 then stores, as candidate image data in the storage unit 19, B-mode image data, of the B-mode image data corresponding to calculated mean square errors equal to or less than a predetermined threshold, which exhibits the largest number of color pixels, and Doppler image data in a corresponding phase. Subsequently, the control processor 18 repeats the above process with respect to the B-mode image data group corresponding to mean square errors equal to or more than the threshold to sequentially store the B-mode image data obtained in the same manner and Doppler image data in corresponding phases as candidate image data in the storage unit 19.

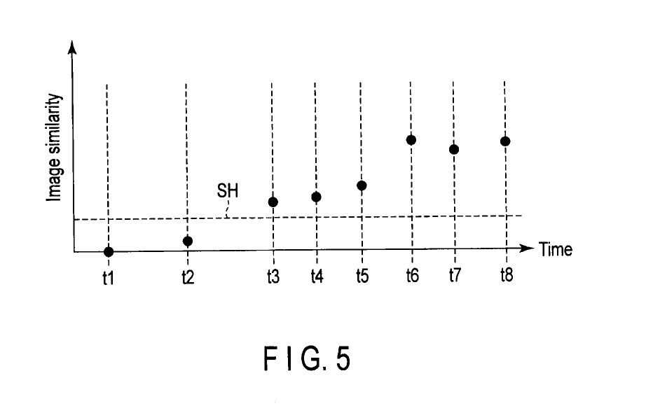

In the case shown in FIG. 4, for example, the control processor 18 calculates, first of all, the mean square errors of the differences between pixels of B-mode image data corresponding to time t1 and B-mode image data corresponding to time t2 to t8. FIG. 5 shows the conceptual view obtained by plotting the obtained mean square errors. The plot at time t1 is shown for the sake of convenience. The corresponding image similarity is the mean square error between B-mode image data corresponding to time t1 and hence is “0”. Assume that in this case, a threshold SH is set, as shown in FIG. 5, as a criterion for determining whether B-mode image data are similar to each other.

Referring to FIG. 5, since the mean square error at time t2 is equal to or less than the threshold, the control processor 18 regards the B-mode image data corresponding to times t1 and t2 as similar image data, and stores, as the first candidate image data in the storage unit 19, B-mode image data, of the B-mode image data at times t1 and t2, which has the largest number of color pixels and Doppler image data in the corresponding phase. The control processor 18 repeats similar processing for the remaining

B-mode image data. That is, the control processor 18 calculates the mean square errors between the B-mode image data, of the remaining B-mode image data, which corresponds to time t3 and the B-mode image data corresponding to times t4 to t8. Assume that in this case, the mean square errors with respect to the B-mode image data corresponding to times t4 and t5 are equal to or less than the threshold SH. In this case, the control processor 18 compares the numbers of color pixels between the B-mode image data corresponding to times t3 to t5, and stores, as the second candidate image data in the storage unit 19, the B-mode image data corresponding to time t5 which has the largest number of color pixels and Doppler image data in the corresponding phase. In addition, the control processor 18 calculates the mean square errors between the B-mode image data, of the remaining B-mode image data, which corresponds to time t6 and the B-mode image data corresponding to times t7 to t8. Assume that in this case, both the mean square errors corresponding to times t7 and t8 are equal to or less than the threshold SH. In this case, the control processor 18 compares the numbers of color pixels between the B-mode image data corresponding to times t6 to t8, and stores, as the third candidate image data in the storage unit 19, the B-mode image data corresponding to time t6 which has the largest number of color pixels and Doppler image data in the corresponding phase. In this manner, the control processor 18 calculates and compares image similarities with respect to all the image data corresponding to the respective points extracted in step S109 and selects candidate image data. In the case shown in FIG. 4, the control processor 18 selects three candidate image data. Finally, the control processor 18 executes processing for displaying the candidate image data (step S111). That is, the control processor 18 outputs the B-mode image data and the Doppler image data which constitute each candidate image data stored in the storage unit 19 to the image combining unit 17. The image combining unit 17 generates display data by combining the input B-mode image data and Doppler image data, and outputs the display data to the monitor 4. The monitor 4 displays candidate images having color Doppler images superimposed on monochrome B-mode images based on the input display data.

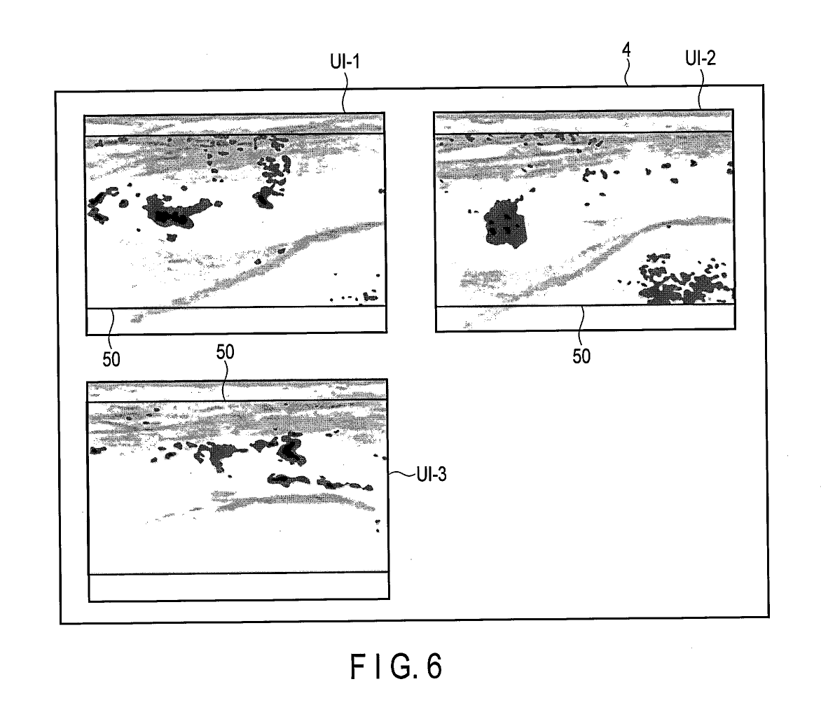

FIG. 6 shows display examples of candidate images. In the case of FIG. 6, three ultrasonic images UI-1, UI-2, and UI-3 as candidate images are simultaneously displayed side by side. In each of the ultrasonic images UI-1, UI-2, and UI-3, low-brightness portions scattered inside the color ROI 50 represent color pixels corresponding to the power of a blood flow.

Note that the monitor 4 may display only one ultrasonic image, and the operator may switch the ultrasonic image displayed on the monitor 4 by operating the input device 3. In addition, the monitor 4 may display a blood flow area or area ratio in a predetermined region in each ultrasonic image, together with the ultrasonic image. A blood flow area is, for example, the number of color pixels in a predetermined region or the value obtained by converting the number of color pixels into an actual area by multiplying the number by a predetermined coefficient. An area ratio is, for example, the value obtained by dividing the number of color pixels in a predetermined region by the total number of pixels in the predetermined region and expressing the quotient in percentage. Note that as a predetermined region, for example, the first region of interest or the second region of interest set in step S105 can be used.

As is obvious from the above description, the control processor 18 functions as a parameter calculation unit which calculates a parameter (the number of color pixels) representing a characteristic of each Doppler image data based on a plurality of Doppler image data, a similarity calculation unit which calculates an image similarity (mean square error) for each combination of B-mode image data and Doppler image data of a plurality of B-mode image data and a plurality of Doppler image data, and an image selection unit which selects a combination (candidate image data) of B-mode image data and Doppler image data which is suitable for diagnosis from a plurality of B-mode image data and a plurality of Doppler image data based on the parameter calculated by the parameter calculation unit and the image similarity calculated by the similarity calculation unit.

The effects of this embodiment will be described.

According to the arrangement of this embodiment, when the operator observes, for example, ultrasonic images (B-mode images and Doppler images) concerning a plurality of slices of a specific region of the subject P and selects an ultrasonic image suitable for diagnosis from the observed images, the ultrasonic diagnostic apparatus automatically selects an ultrasonic image suitable for diagnosis by the operation shown in the flowchart of FIG. 2. This can reduce burden on the operator.

In addition, considering image similarities can prevent similar ultrasonic images from being redundantly selected, and can present wide variations of ultrasonic images to the user. This can prevent diagnosis errors, and, for example, allows the user to select an ultrasonic image suitable for diagnosis by comparing and studying a small number of ultrasonic images when narrowing down presented ultrasonic. images.

In addition, excluding ultrasonic images in phases in which motion is large (an average velocity value is large) in step S108 can prevent the selection of an ultrasonic image which is mixed with motion artifacts and unsuitable for diagnosis.

Furthermore, since the numbers of color pixels to be used for the selection of an ultrasonic image are calculated in the second region of interest set based on B-mode image data, the numbers of color pixels in portions which do not contribute to diagnosis, e.g., a blood flow in a normal blood vessel, do not easily mix. This can improve the accuracy of the selection of an ultrasonic image.

Note that images whose similarity is to be calculated are not limited to morphological images. For example, it is possible to calculate the similarity between blood flow images, contrast-enhanced blood vessel images, and elastography images indicating the spatial distribution of elastic moduli of a tissue.

Although a Doppler image has been exemplified as a target image to be finally selected, a contrast-enhanced blood vessel image may be a target image. In this case, a contrast-enhanced blood vessel image whose number of pixels having contrast brightness equal to or more than a threshold is equal to or more than a predetermined number is selected as a candidate image.

In addition, as shown in FIG. 4, (A) it is possible to use a B-mode image in the phase nearest to phase t1 of the Doppler image acquired first after the start of ultrasonic scanning as a reference image for similarity calculation and calculate the similarity (least square error) between the reference image and a subsequent image, or (B) it is possible to use a B-mode image in the phase nearest to phase t2 of the Doppler image whose number of color pixels exceeds a threshold first after the start of ultrasonic scanning as a reference image for similarity calculation and calculate the similarity (least square error) between the reference image and a subsequent image.

As described above, the apparatus selects a Doppler image by using the similarity between morphological images. In other words, the apparatus excludes a Doppler image obtained at a time near the scanning time of a morphological image exhibiting a high similarity from display targets. This is a very novel technical idea.

### Second Embodiment

The second embodiment will be described.

The first embodiment has exemplified the case of calculating the numbers of color pixels only in the second region of interest set in B-mode image data, excluding unsuitable images based on an average velocity or velocity variance, and narrowing down a plurality of obtained image data to candidate image data based on the image similarities calculated based on the brightness of B-mode image data. The second embodiment will exemplify, as a simpler technique, the case of calculating the numbers of color pixels in an entire color ROI (first region of interest), excluding unsuitable image data based on only the calculated numbers of color pixels, segmenting an image data group into a plurality of regions based on the numbers of color pixels of the remaining image data, and extracting a limited number of candidate image data from the respective regions.

Note that since the arrangement of an ultrasonic diagnostic apparatus according to this embodiment is the same as that described in the first embodiment with reference to FIG. 1, a description of this will be omitted.

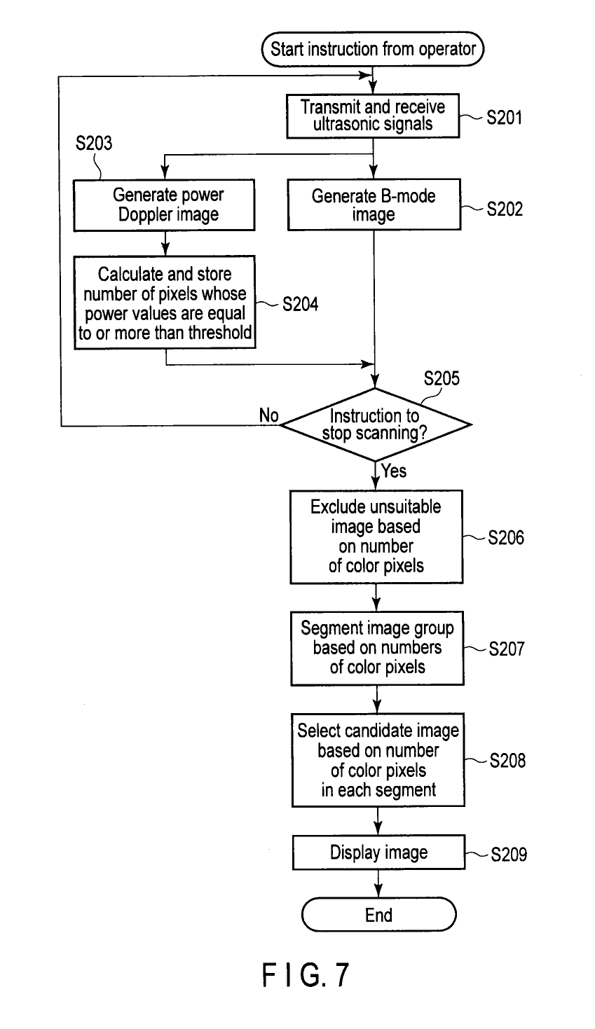

FIG. 7 is a flowchart showing the operation of the ultrasonic diagnostic apparatus according to the second embodiment. Of the operations indicated by this flowchart, the operations in steps S204 and S206 to S208 are implemented by making a control processor 18 execute the analysis program stored in a storage unit 19.

Upon receiving a start instruction from the operator, an ultrasonic probe 2 generates an ultrasonic signal into a subject P as in step S101 (step S201). An image generation unit 15 generates B-mode image data as in step S102 (step S202), and generates Doppler image data as in step S103 (step S203). The image generation unit 15 stores the B-mode image data and the Doppler image data generated in steps S202 and S203 in the storage unit 19 in a form that enables the discrimination of phases of image generation.

The control processor 18 then calculates the total number of color pixels having power values equal to or more than a predetermined threshold and contained in the predetermined first region of interest (step S204). The control processor 18 stores the calculated number of color pixels in association with the Doppler image data generated in step S203 and stored in the storage unit 19.

After steps S202 and S204, the control processor 18 determines, as in step S107, whether the operator has input an instruction to stop scanning (step S205). If the operator has input no instruction (NO in step S205), the process returns to step S201 to repeat steps S201 to S204.

When the operator inputs an instruction to stop scanning by operating an input device 3 (YES in step S205), the control processor 18 executes processing for selecting image data suitable for diagnosis from the plurality of B-mode image data and the plurality of Doppler image data sequentially stored in repeatedly executed steps S202 and S203 (steps S206 to S208).

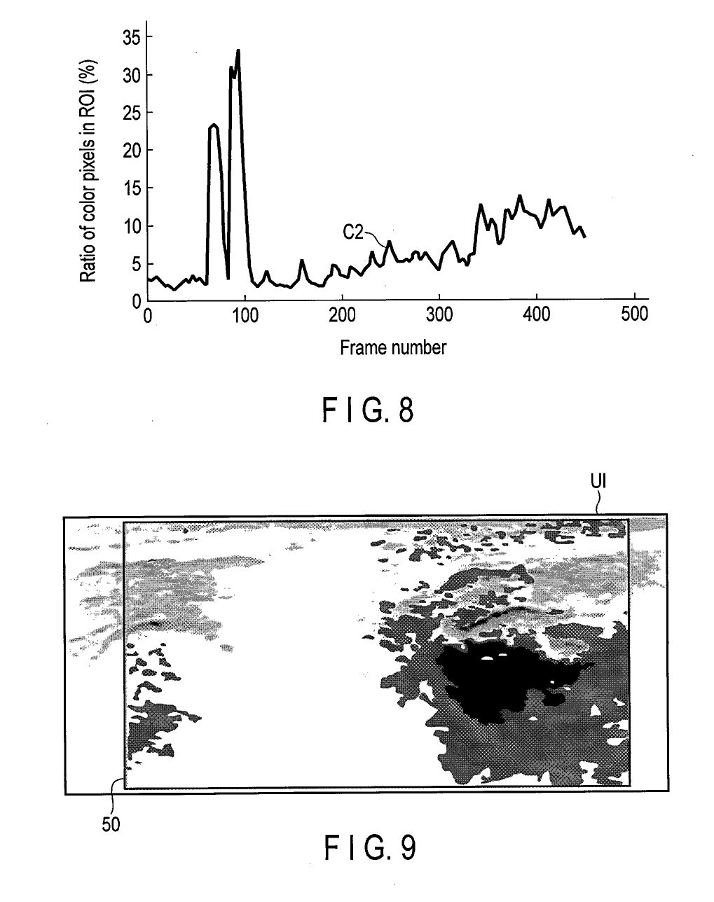

First of all, the control processor 18 generates a time-area curve (plotting of the numbers of color pixels in the respective phases) based on the numbers of color pixels calculated in step S204, and excludes image data unsuitable for diagnosis based on the curve (step S206). FIG. 8 shows an example of a time-area curve. Referring to FIG. 8, the abscissa represents the frame numbers assigned in the order of phases, and the ordinate represents the ratios of color pixels contained in the first region of interest (each value expressed in percentage which is obtained by dividing the number of color pixels calculated in step S204 by the total number of pixels in the first region of interest). The steep peaks appearing near frame numbers 60 to 100 on a time-area curve C2 shown in FIG. 8 originate from motion artifacts.

FIG. 9 shows an example of an ultrasonic image (B-mode image+Doppler image) in which the motion artifacts are depicted. As is obvious from comparison with FIG. 6, motion artifacts (low-brightness portions) appear over a wide range in a color ROI 50. An ultrasonic image UI mixed with motion artifacts (to be referred to as noise image data hereinafter) cannot be used for diagnosis. This embodiment therefore excludes such noise image data from candidate targets. For example, the control processor 18 detects each point as a peak on the plot in FIG. 8 such that the difference between the point and each of the left and right adjacent points is equal to or more than a predetermined value, and excludes B-mode image data and

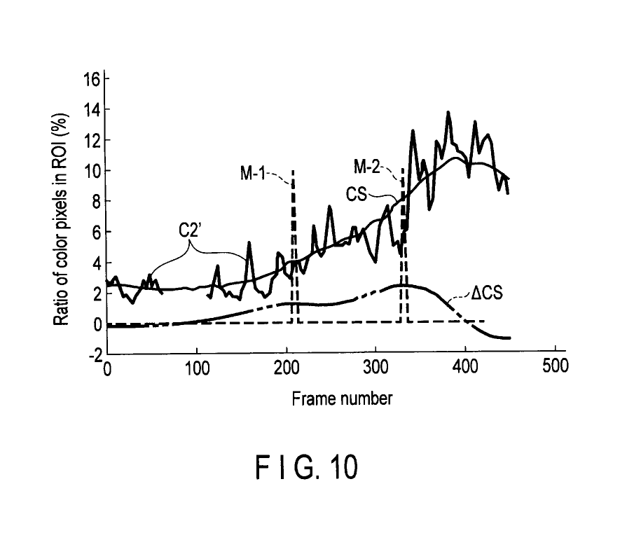

Doppler image data corresponding to all the crests including the detected peaks as noise image data. FIG. 10 shows a time-area curve C2′ after the exclusion of noise image data obtained in this manner.

After step S206, the control processor 18 segments an image data group based on the numbers of color pixels represented by the time-area curve C2′ (step S207). In this processing, the control processor 18 observes a temporal change in the time-area curve C2′, and regards portions where changes are small as similar slices, while regarding portions where changes are large as portions where the slice position has changed, thereby segmenting the image data group into a predetermined number of segments.

Specific processing in step S207 will be described. First of all, the control processor 18 performs smoothing processing for the temporal area curve C2′. FIG. 10 shows an example of a smoothed time-area curve CS. The control processor 18 then obtains a differential curve ACS obtained by temporal differentiation of the smoothed temporal area curve CS. In this case, in order to obtain a stable result, the control processor 18 may perform smoothing processing in addition to temporal differentiation. A point where the temporal differential curve ACS exhibits a maximal value can be regarded as a point where the temporal change curve C2′ has greatly changed. For this reason, the control processor 18 detects the maximal values of the temporal differential curve ACS. In this case, FIG. 10 shows maximal value detection points M. In the case of FIG. 10, the control processor 18 detects two maximum value detection points M-1 and M-2. The control processor 18 segments an image data group by using the maximal value detection points M detected in this manner as delimiter positions for temporal regions. In the case shown in FIG. 10, for example, the control processor 18 segments the image data group into B-mode image data and Doppler image data ranging from the frame number 0 to a frame number less than the frame number at the maximal value detection point M-1, B-mode image data and Doppler image data ranging from a frame number equal to or more than the frame number at the maximal value detection point M-1 to a frame number less than the frame number at the maximal value detection point M-2, and B-mode image data and Doppler image data corresponding to frame numbers equal to or more than the frame number at the maximal value detection point M-2.

Note that in the case of FIG. 10, only two maximal values are obtained. In some cases, however, many maximal values are obtained. For this reason, the maximum number of segments may be determined in advance. If the number of segments delimited by obtained many maximal values exceeds this maximum number of segments, a predetermined number (e.g., maximum number of segments—1) of maximal values to be used for delimiting may be selected from the many maximal values in descending order of values on the temporal differential curve ACS.

After step S207, the control processor 18 selects candidate image data in the respective segments set in step S207 (step S208). More specifically, the control processor 18 extracts, based on the time-area curve C2, all the points where the numbers of color pixels are maximal as in step S109 in the first embodiment. In addition, the control processor 18 extracts a predetermined number of points (e.g., one point) from the extracted points in descending order of the numbers of color pixels in the respective segments. B-mode image data and Doppler image data corresponding to each point extracted in this manner become candidate image data.

Finally, the control processor 18 executes processing for displaying an ultrasonic image (B-mode image+Doppler image) concerning candidate image data as in step S111 (step S209). A plurality of ultrasonic images may be displayed in chronological order or in descending order of the numbers of color pixels. Alternatively, all or a predetermined number of ultrasonic images may be simultaneously displayed side by side. In addition, it is possible to display each ultrasonic image together with a blood flow area or area ratio in a predetermined region in the ultrasonic image.

The effects of this embodiment will be described.

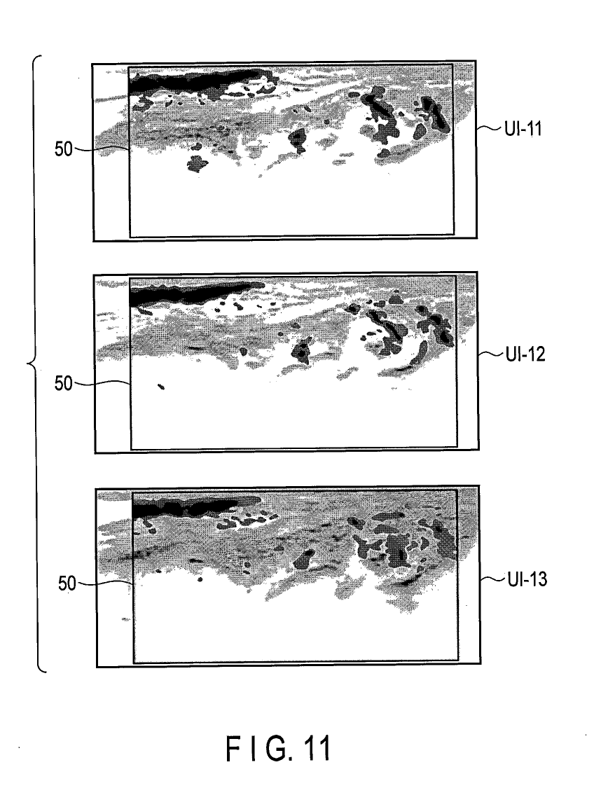

FIG. 11 shows an example of displaying ultrasonic images UI-11, UI-12, and UI-13 based on B-mode image data and Doppler image data corresponding to three frame numbers in descending order of the numbers of color pixels on the time-area curve C2′ after step S206. As is obvious from FIG. 11, although a plurality of ultrasonic images are displayed, they are similar images. That is, they add no new diagnostic information. This is because, as is obvious from the time-area curve C2′ exemplified by FIG. 10, only image data in a time region near the last (in a phase corresponding to a large frame number) are selected. Each image data in this time region corresponds to a normal blood flow, and hence the number of color pixels in the first region of interest is large.

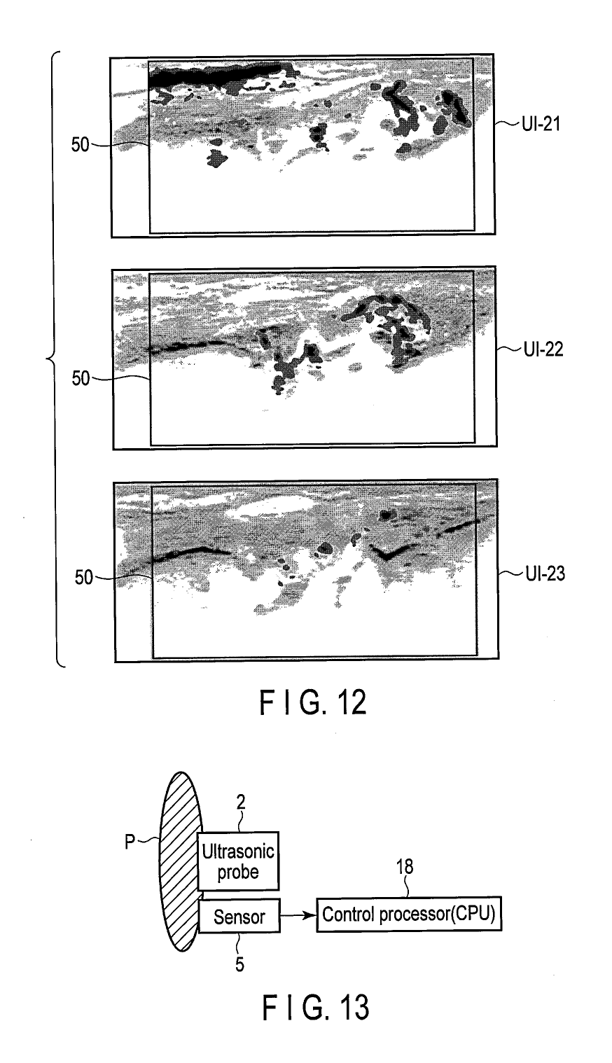

On the other hand, FIG. 12 shows an example of displaying ultrasonic images UI-21, UI-22, and UI-23 based on candidate image data, each having the largest number of color pixels, selected, upon segmentation of the image data group in step S207, from the respective segments according to maximal value detection points M. It is obvious from this example that appropriate candidate images reflecting an inflammatory blood flow can be displayed in a region (a middle portion in the color ROI 50) where almost no normal blood flow (the low-brightness portion on the upper left portion in the color ROI 50 in the ultrasonic image UI-21) exists.

As described above, according to this embodiment, it is possible to select rich variations of candidate image data from an image data group accurately with a simpler arrangement and a smaller amount of calculation. Calculating the numbers of color pixels in the entire first region of interest will increase the possibility of extracting image data dominantly depicting a normal blood vessel which does not contribute to diagnosis. However, segmenting an image data group based on the degree of change in the number of color pixels enables selection of candidate image data even from a region with a small number of color pixels. This makes it possible to display, on the monitor 4, even an ultrasonic image depicting only a small inflammatory blood flow which is useful for diagnosis.

In addition, the same effects as those of the first embodiment can be obtained.

### Third Embodiment

The third embodiment will be described.

The first and second embodiments are configured to exclude image data containing large motion artifacts as image data unsuitable for diagnosis based on the temporal change in average velocity or the number of color pixels in the first region of interest. In the third embodiment, an ultrasonic probe 2 is provided with a sensor for detecting information concerning the position, posture, or velocity of the ultrasonic probe 2 to exclude image data unsuitable for diagnosis by using the information detected by the sensor. In addition, this embodiment narrows down candidate image data by using the information detected by the sensor.

The arrangement of the ultrasonic diagnostic apparatus according to this embodiment is almost the same as that described with reference to FIG. 1 in the first embodiment. Note however that the ultrasonic diagnostic apparatus according to this embodiment differs from that in the first embodiment in that it includes a sensor 5 connected to a control processor 18, as shown in FIG. 13.

The sensor 5 detects information concerning the position, posture, or velocity of the ultrasonic probe 2, and outputs the detection result to the control processor 18.

It is possible to use, for example, a magnetic sensor as the sensor 5. In this case, a transmitter which forms a magnetic field having a predetermined strength is placed near a subject P, and the sensor 5 as the magnetic sensor is attached to the ultrasonic probe 2. The sensor 5 detects the position (x, y, z) and posture (θx, θy, θz) of the ultrasonic probe 2 in the three-dimensional coordinate space (X, Y, Z) defined by the X-, Y-, and Z-axes with the transmitter being the origin. In this case, x represents the position of the ultrasonic probe 2 on the X-axis, y represents the position of the ultrasonic probe 2 on the Y-axis, and z represents the position of the ultrasonic probe 2 on the Z-axis. In addition, θx represents the rotational angle of the ultrasonic probe 2 centered on the X-axis, θy represents the rotational angle of the ultrasonic probe 2 centered on the Y-axis, and θz represents the rotational angle of the ultrasonic probe 2 centered on the Z-axis. The sensor 5 may further include a unit for calculating the velocity (vx, vy, vz) of the ultrasonic probe 2 based on a temporal change in position (x, y, z). In this case, vx represents the velocity of the ultrasonic probe 2 in the X-axis direction, vy represents the velocity of the ultrasonic probe 2 in the Y-axis direction, and vz represents the velocity of the ultrasonic probe 2 in the Z-axis direction.

It is also possible to use, for example, a triaxial acceleration sensor as the sensor 5. Even when the sensor 5 which is an acceleration sensor is attached to the ultrasonic probe 2, it is possible to calculate the posture (θx, θy, θz) and velocity (vx, vy, vz) of the ultrasonic probe 2 based on the triaxial acceleration detected by the sensor 5.

In addition to the above sensor, various types of sensors can be used as the sensor 5, including an optical sensor which optically detects the position and posture of the ultrasonic probe 2.

This embodiment uses one of the above sensors or a combination of a plurality of sensors to form the sensor 5 which detects the position (x, y, z), posture (θx, θy, θz) and velocity (vx, vy, vz) of the ultrasonic probe 2.

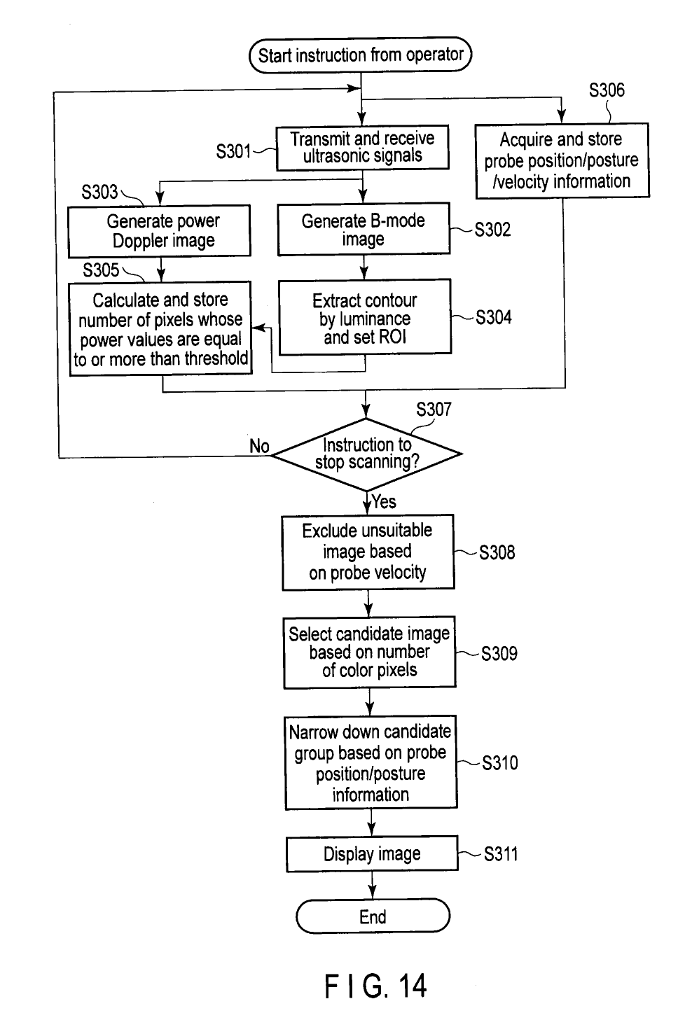

FIG. 14 is a flowchart showing the operation of the ultrasonic diagnostic apparatus according to the third embodiment.

Of the operations indicated by this flowchart, the operations in steps S305, S306, and S308 to S310 are implemented by making the control processor 18 execute the analysis program stored in a storage unit 19.

Upon receiving a start instruction from the operator, the ultrasonic probe 2 generates an ultrasonic signal into the subject P as in step S101 (step S301). An image generation unit 15 generates B-mode image data as in step S102 (step S302), and generates Doppler image data as in step S103 (step S303). The image generation unit 15 stores the B-mode image data and the Doppler image data generated in steps S302 and S303 in the storage unit 19 in a form that enables the discrimination of phases of image generation.

The control processor 18 then performs segmentation and sets a region of interest (second region of interest) based on the B-mode image data obtained in step S302 by using the same technique as that in step S105 (step S304). In addition, the control processor 18 calculates the number of color pixels of a Doppler signal contained in the second region of interest set in step S304 in the Doppler image data generated in step S303 by using the same technique as that in step S106 (step S305). The control processor 18 stores the calculated number of color pixels in association with the Doppler image data generated in step S303 and stored in the storage unit 19.

In this embodiment, the control processor 18 executes step S306 concurrently with steps S301 to S305. That is, the control processor 18 acquires information concerning the position (x, y, z), posture (θx, θy, θz), and velocity (vx, vy, vz) of the ultrasonic probe 2 detected by the sensor 5 from the sensor 5, and stores the information in the storage unit 19 in a form that enables the discrimination of a phase at the time of acquisition.

After steps S304, S305, and S306, the control processor 18 determines, as in step S107, whether the operator has input an instruction to stop scanning (step S307). If the operator has input no instruction (NO in step S307), the process repeats steps S301 to S306.

When the operator inputs an instruction to stop scanning by operating an input device 3 (YES in step S307), the control processor 18 executes processing for selecting image data suitable for diagnosis from the plurality of B-mode image data and the plurality of Doppler image data sequentially stored in repeatedly executed steps S302 and S303 (steps S308 to S310).

First of all, the control processor 18 excludes image data unsuitable for diagnosis based on the velocity (vx, vy, vz) in each phase stored in the storage unit 19 (step S308).

More specifically, the control processor 18 sequentially reads the velocity (vx, vy, vz) in each phase. If the value is equal to or more than a predetermined threshold, the control processor 18 excludes B-mode image data and Doppler image data corresponding to the phase from selection targets. This threshold indicates the boundary between the velocity at which a motion artifact unsuitable for diagnosis appears in Doppler image data and otherwise, and may be obtained experimentally, empirically, or theoretically. This makes it possible to exclude, from choices, image data in which a motion artifact seems to have occurred due to the large movement of the probe.

The control processor 18 then selects a plurality of candidate image data based on the numbers of color pixels as in step S109 (step S309).

Subsequently, the control processor 18 narrows down candidate image data based on the position (x, y, z) and posture (θx, θy, θz) of the ultrasonic probe 2 (step S310).

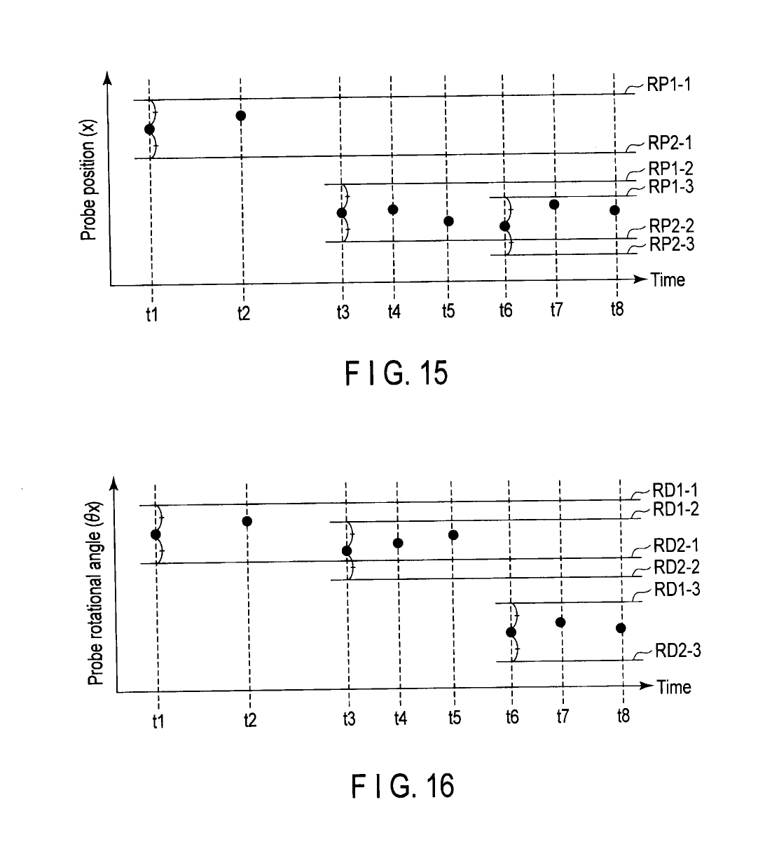

More specifically, first of all, the control processor 18 reads the positions (x, y, z) and postures (θx, θy, θz) in phases corresponding to a plurality of candidate image data selected in step S309 from the storage unit 19. FIGS. 15 and 16 are conceptual views each showing plotting of the read positions (x, y, z) and postures (θx, θy, θz).

FIG. 15 is a graph plotting the X-coordinates x at the positions (x, y, z) of phases corresponding to the plurality of candidate image data selected in step S309. First of all, consider the position at time t1 corresponding to the earliest phase as a reference. The control processor 18 sets reference positions RP1 and RP2 shifted from the position at time t1 by a predetermined threshold in the positive/negative direction, and specifies a phase having a plot between the reference positions RP1 and RP2. This threshold is set to a value that can regard a plot, within the range defined by the reference positions RP1 and RP2 as the upper and lower limits, as being located at the same position as that of a reference plot. Referring to FIG. 15, the reference positions RP1 and RP2 set at this time are written as reference positions RP1-1 and RP2-1. Referring to FIG. 15, a plot appears at only time t2 other than time t1 between the reference positions RP1-1 and RP2-1. The control processor 18 performs similar analysis using the reference positions RP1 and RP2 also at the Y-coordinates y and the Z-coordinates z to specify phases having plots between the reference positions RP1 and RP2 at all the X-coordinates x, Y-coordinates y, and Z-coordinates z. Assume that as a result of such analysis, in the case shown in FIG. 15, only the position (x, y, z) corresponding to time t2 is specified as a position almost equal to the position (x, y, z) corresponding to time t1.

Subsequently, the control processor 18 analyzes the posture (θx, θy, θz) of the probe. FIG. 16 is a conceptual view plotting the rotational angles θx about the X-axis at times t1 to t8 shown in FIG. 15. The control processor 18 sets reference angles RD1 and RD2 shifted from the rotational angle at time t1 by a predetermined threshold in the positive/negative direction. This threshold is set to a value that can regard a plot, within the range defined by the reference angles RD1 and RD2 as the upper and lower limits, as having the same posture as that of a reference plot. Referring to FIG. 16, the reference angles RD1 and RD2 set at this time are written as reference angles RD1-1 and RD2-1. Referring to FIG. 16, the plots corresponding to times t2 to t5, other than the plot corresponding to time t1, fall within the range from the reference angle RD1-1 to the reference angle RD2-1. The control processor 18 performs similar analysis using the reference angles RD1 and RD2 also at the rotational angles θy and θz to specify phases having plots between the reference angles RD1 and RD2 at all the rotational angles θx, θy, and θz. Assume that as a result of such analysis, in the case shown in FIG. 16, the postures (θx, θy, θz) corresponding to times t2 to t5 are specified as postures almost equal to the posture (θx, θy, θz) corresponding to time t1.

Finally, the control processor 18 specifies a phase common to the phases specified by the analysis using the positions (x, y, z) and the phases specified by the analysis using the postures (θx, θy, θz), and selects one of Doppler image data corresponding to the specified phase and the reference phase which has the largest number of color pixels calculated in step S305 and corresponding B-mode image data as candidate image data. In the case shown in FIGS. 15 and 16, for example, the control processor 18 selects one data, of B-mode image data and Doppler image data corresponding to time t2 common to time t2 specified by the analysis using the positions (x, y, z) and times t2 to t5 specified by the analysis using the postures (θx, θy, θz) and reference time t1, which has the largest number of color pixels calculated in step S305 as candidate image data.

The control processor 18 repeats similar analysis and candidate image data selection for phases except for the phase common to the phases specified by the analysis using the positions (x, y, z) and the phases specified by the analysis using the postures (θx, θy, θz) and the reference phase. For example, in the case shown in FIGS. 15 and 16, times t3 to t8 are targets for the next analysis and selection. In the case of FIG. 15, the control processor 18 newly sets reference positions RP1 and RP2 with reference to the plot at time t3. Referring to FIG. 15, the reference positions RP1 and RP2 set at this time are written as reference positions RP1-2 and RP2-2. Referring to FIG. 15, plots appear at times t4 to t8 other than time t3 between the reference positions RP1-2 and RP2-2. The control processor 18 performs similar analysis using the reference positions RP1 and RP2 also at the Y-coordinates y and the Z-coordinates z to specify phases having plots between the reference positions RP1 and RP2 at all the X-coordinates x, Y-coordinates y, and Z-coordinates z. Assume that as a result of such analysis, in the case shown in FIG. 15, the positions (x, y, z) corresponding to times t4 to t8 are specified as positions almost equal to the position (x, y, z) corresponding to time t3.

Subsequently, the control processor 18 analyzes the posture (θx, θy, θz) of the probe. In the case of FIG. 16, the control processor 18 sets reference angles RD1 and RD2 shifted by a predetermined threshold in the positive/negative direction with reference to the rotational angle θx at time t3. Referring to FIG. 16, the reference angles RD1 and RD2 set at this time are written as reference angles RD1-2 and RD2-2. Referring to FIG. 16, the plots corresponding to times t4 and t5, other than the plot corresponding to time t3, fall within the range from the reference angle RD1-2 to the reference angle RD2-2.

The control processor 18 performs similar analysis using the reference angles RD1 and RD2 also at the rotational angles θy and θz to specify phases having plots between the reference angles RD1 and RD2 at all the rotational angles θx, θy, and θz. Assume that as a result of such analysis, in the case shown in FIG. 16, the postures (θx, θy, θz) corresponding to times t4 and t5 are specified as postures almost equal to the posture (θx, θy, θz) corresponding to time t3.

Finally, the control processor 18 selects one data, of Doppler image data corresponding to times t4 and t5 common to times t4 to t8 specified by the analysis using the positions (x, y, z) and times t4 and t5 specified by the analysis using the postures (θx, θy, θz) and reference time t3, which has the largest number of color pixels calculated in step S305 and the corresponding B-mode image data as the second candidate image data.

Subsequently, the control processor 18 repeats similar analysis and candidate image data selection for phases except for the phases common to the phases specified by the analysis using the positions (x, y, z) and the phases specified by the analysis using the postures (θx, θy, θz) and the reference phase. For example, in the case shown in FIGS. 15 and 16, times t6 to t8 are targets for the next analysis and selection. The control processor 18 selects the third candidate image data from B-mode image data and Doppler image data corresponding to these phases. The reference positions RP1 and RP2 used to select the third candidate image data are written as reference positions RP1-3 and RP2-3 in FIG. 15. In addition, the reference angles RD1 and RD2 used to select the third candidate image data are written as reference angles RD1-3 and RD2-3 in FIG. 16. The control processor 18 executes such processing until no phase as a target for analysis and selection is left.

After step S310, the control processor 18 executes processing for displaying a plurality of ultrasonic images (B-mode images+Doppler images) concerning a plurality of candidate image data narrowed down in step S310 as in step S111 (step S311). A plurality of ultrasonic images may be displayed in chronological order or in descending order of the numbers of color pixels.

Alternatively, all or a predetermined number of ultrasonic images may be simultaneously displayed side by side. In addition, it is possible to display each ultrasonic image together with a blood flow area or area ratio in a predetermined region in the ultrasonic image.

With the arrangement of the third embodiment described above, it is possible to obtain the same effects as those of the first embodiment.

### Fourth Embodiment

The fourth embodiment will be described.

This embodiment will disclose an image processing apparatus which reads moving data or a series of still image data stored in the ultrasonic diagnostic apparatus and automatically selects image data.

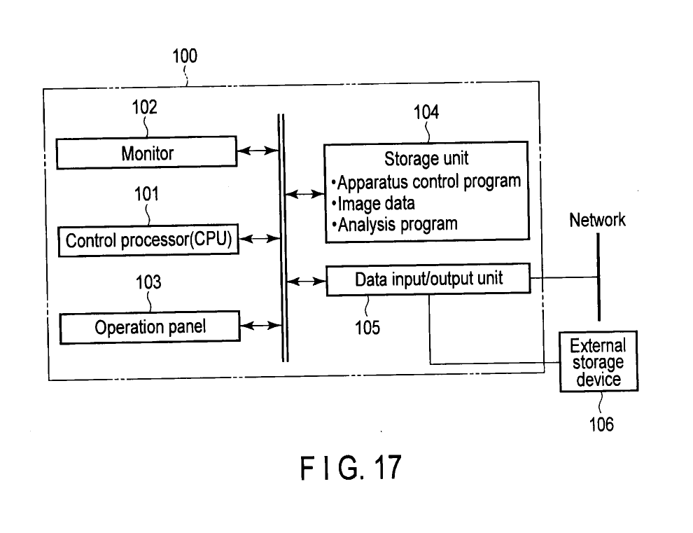

FIG. 17 is a block diagram showing the arrangement of the main part of an image processing apparatus according to this embodiment.

A main body 100 of this image processing apparatus includes a control processor 101, a monitor 102, an operation panel 103, a storage unit 104, and a data input/output unit 105.

The control processor 101 is mainly constituted by, for example, a CPU and memories such as a ROM and a RAM, and functions as a control unit which controls the operation of the apparatus main body 100. The control processor 101 reads out control programs for executing image generation, display, and the like from a storage unit 19, and executes computation, control, and the like concerning various types of processing.

The monitor 102 selectively displays the ultrasonic images based on the B-mode image data and Doppler image data obtained by the ultrasonic diagnostic apparatus, various types of graphical user interfaces, and the like.

The operation panel 103 includes various types of switches, buttons, a trackball, a mouse, and a keyboard which are used to input various types of instructions from an operator.

The storage unit 104 stores various types of control programs and analysis programs. The storage unit 104 also functions to hold the image data and numerical data input by the image processing apparatus.

The data input/output unit 105 connects a network such as a LAN to the apparatus main body 100. An ultrasonic diagnostic apparatus and an information processing system in a hospital are connected to this network. The data input/output unit 105 also connects an external storage device 106 to the apparatus main body 100. The data input/output unit 105 transmits and receives data to and from the apparatus connected to a network and the external storage device 106.

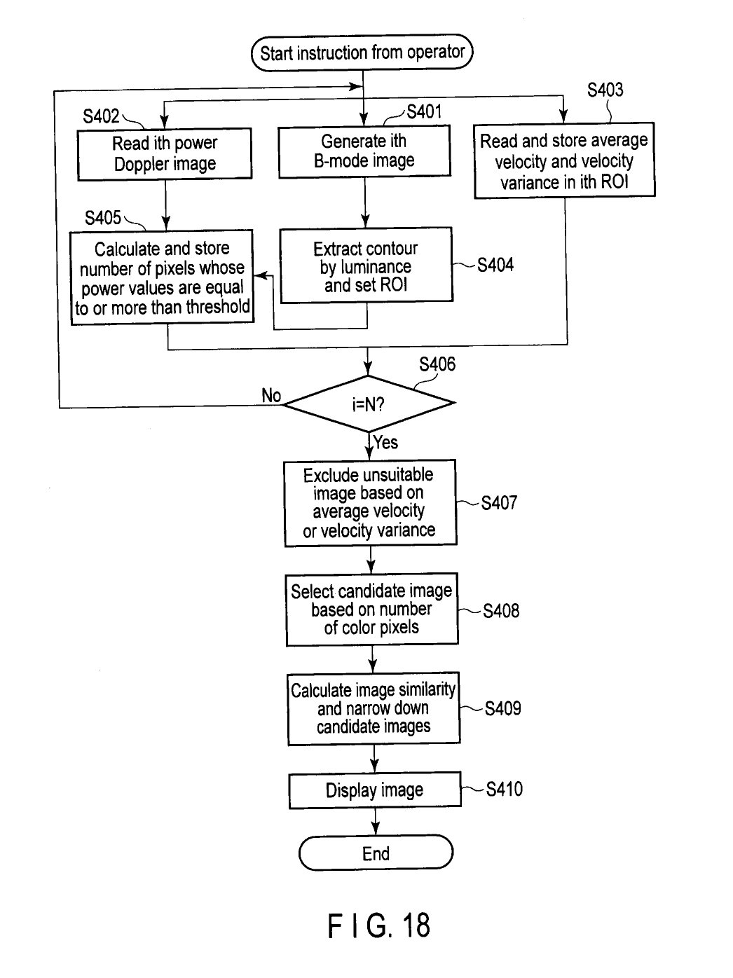

An operation procedure in this embodiment will be described with reference to the flowchart of FIG. 18. A basic operation procedure is the same as that in the first embodiment. Note however that this image processing apparatus differs from that in the first embodiment in that it reads B-mode image data and Doppler image data from a network connected to the data input/output unit 105 or the external storage device 106 and processes the data without performing ultrasonic transmission/reception or generating any image data.

Assume that in the following description, the ultrasonic diagnostic apparatus connected to a network has already executed the processing in steps S101 to S104 in the first embodiment, and has stored, in the external storage device 106, the resultant B-mode image data and Doppler image data corresponding to phases 1 to N (N is an integer), velocity information and velocity variance information (e.g., an average velocity value, an average variance value, and a variance value of velocities) in the first region of interest in each Doppler image data in a form that enables the discrimination of phases of image generation.

When the operator inputs an instruction to start processing by operating the operation panel 103, the control processor 101 reads B-mode image data corresponding to the ith phase from the external storage device 106 via the data input/output unit 105 and stores the read data in the storage unit 104 (step S401). The control processor 101 then reads Doppler image data corresponding to the ith phase from the external storage device 106 and stores the read data in the storage unit 104 (step S402). The control processor 101 also reads velocity information and velocity variance information in the first region of interest corresponding to the ith phase from the external storage device 106 and stores the read information in the storage unit 104 (step S403). Note that i represents the value of a counter which is generated by the control processor 101 in its memory and is an integer equal to or more than 1 and equal to or less than N. The counter i is given as i=1 at the start of issuing an instruction to start processing, and is incremented by one every time steps S401 to S403 are executed.

Subsequently, the control processor 101 sets a region of interest (second region of interest) by using the same technique as that in step S105 based on the B-mode image data obtained in step S401 (step S404). Upon setting the second region of interest, the control processor 101 calculates the number of color pixels of a Doppler signal contained in the second region of interest set in step S404 in the Doppler image data read in step S402 by using the same technique as that in step S106 (step S405). The control processor 101 stores the calculated number of color pixels in the storage unit 104 in association with the Doppler image data read and stored in the storage unit 104 in step S402.

After step S405, the control processor 101 determines whether the counter i has reached N (step S406). If the counter i has not reached N (NO in step S406), the control processor 101 increments the counter i by one to execute steps S401 to S405 again.

When the counter i has reached N (YES in step S406), the control processor 101 executes processing for selecting candidate image data suitable for diagnosis from a plurality of B-mode image data and a plurality of Doppler image data sequentially stored in repeatedly executed steps S401 and S402 (steps S407 to S409). The control processor 101 then displays the selected candidate image data on the monitor 102 (step S410). Since steps S407 to S410 are the same as steps S108 to S111, a description of them will be omitted.

When the operator selects an image to be left for a final report from a plurality of image data temporarily stored at the time of examination using the ultrasonic diagnostic apparatus upon reviewing image data thereafter, the image processing apparatus according to this embodiment can reduce burden on the operator to allow him/her to select image data useful for diagnosis within a shorter time. This apparatus is especially useful when an examiner and an interpreter of image data are different persons. The examiner need not select image data useful for diagnosis by himself/herself, and hence can focus on scanning. In addition, the interpreter can select image data useful for diagnosis by efficiently checking a series of image data, stored by the examiner, in a short time. This makes it possible to eliminate the possibility of diagnosis errors due to subjective selection of image data by the examiner, and allows the interpreter to provide reliable diagnostic information.

The fourth embodiment also has the same effects as those of the first embodiment.

### Fifth Embodiment

The fifth embodiment will be described.

In this embodiment, as in the second embodiment, the image processing apparatus shown in FIG. 17 differs from that in the fourth embodiment in that it calculates the number of color pixels in an entire color ROI (first region of interest), excludes unsuitable image data based on only the calculated number of color pixels, segments an image data group into a plurality of regions based on the numbers of color pixels of the remaining image data, and extracts a limited number of candidate image data from the respective regions.

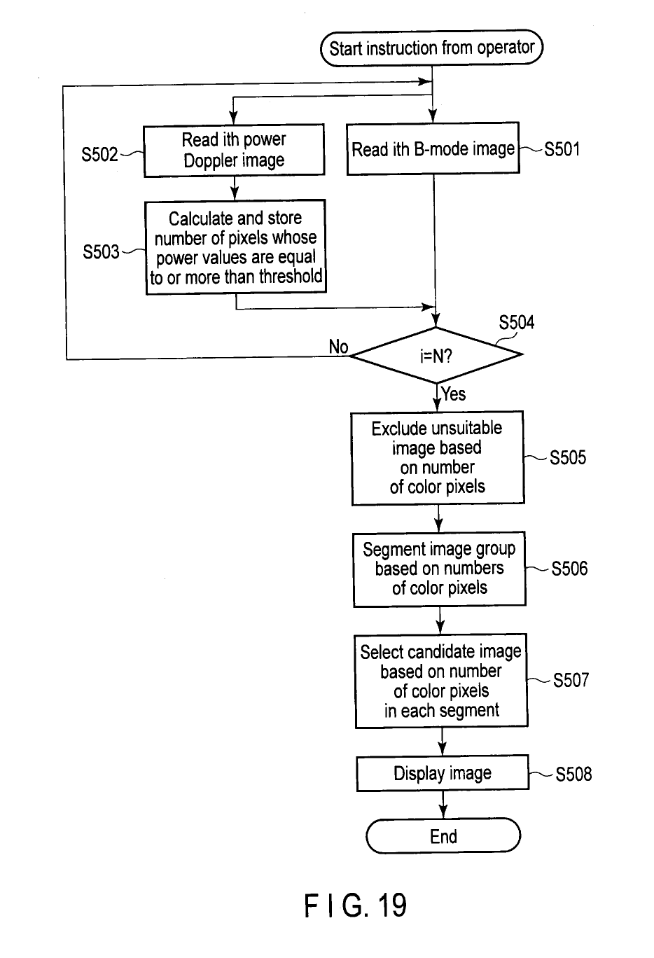

An operation procedure in this embodiment will be described with reference to the flowchart of FIG. 19. A basic operation procedure is the same as that in the second embodiment. However, this image processing apparatus differs from that in the second embodiment in that it reads B-mode image data and Doppler image data from a network connected to a data input/output unit 105 or an external storage device 106 and processes the data without performing ultrasonic transmission/reception or generating any image data.

Assume that in the following description, as in the fourth embodiment, the external storage device 106 stores in advance B-mode image data and Doppler image data corresponding to phases 1 to N and velocity information and velocity variance information (average velocity values, average variance values, variance values of velocities, and the like) concerning the inside of the first region of interest in the respective Doppler image data in a form that enables the discrimination of phases of image generation.

When the operator issues an instruction to start processing by operating an operation panel 103, a control processor 101 reads B-mode image data corresponding to the ith phase from the external storage device 106 and stores the data in a storage unit 104 as in step S401 (step S501). As in step S402, the control processor 101 reads Doppler image data corresponding to the ith phase from the external storage device 106 and stores the data in the storage unit 104 (step S502).

Subsequently, the control processor 101 calculates the total number of color pixels having power values equal to or more than a preset threshold and contained in the first region of interest by the same technique as that in step S204 (step S503). The control processor 101 stores the calculated number of color pixels in the storage unit 104 in association with the Doppler image data stored in the storage unit 104 in step S502.

After step S503, the control processor 101 determines whether the counter i has reached N (step S504). If the counter i has not reached N (NO in step S504), the control processor 101 increments the counter i by one to execute steps S501 to S503 again.

When the counter i has reached N afterward (YES in step S504), the control processor 101 executes processing for selecting candidate image data suitable for diagnosis from the plurality of B-mode image data and the plurality of Doppler image data sequentially stored in repeatedly executed steps S501 and S502 (steps S505 to S507). The control processor 101 displays the selected candidate image data on the monitor 102 (step S508). Since steps S505 to S508 are the same as steps S206 to S209, a description of them will be omitted.

The image processing apparatus according to this embodiment can obtain the same effects as those of the second and fourth embodiments.

### Sixth Embodiment

The sixth embodiment will be described.

The image processing apparatus shown in FIG. 17 in this embodiment as in the third embodiment differs from that in the fourth embodiment in that it excludes image data unsuitable for diagnosis by using information concerning the position, posture, or velocity of the ultrasonic probe and narrows down candidate image data by using the information.

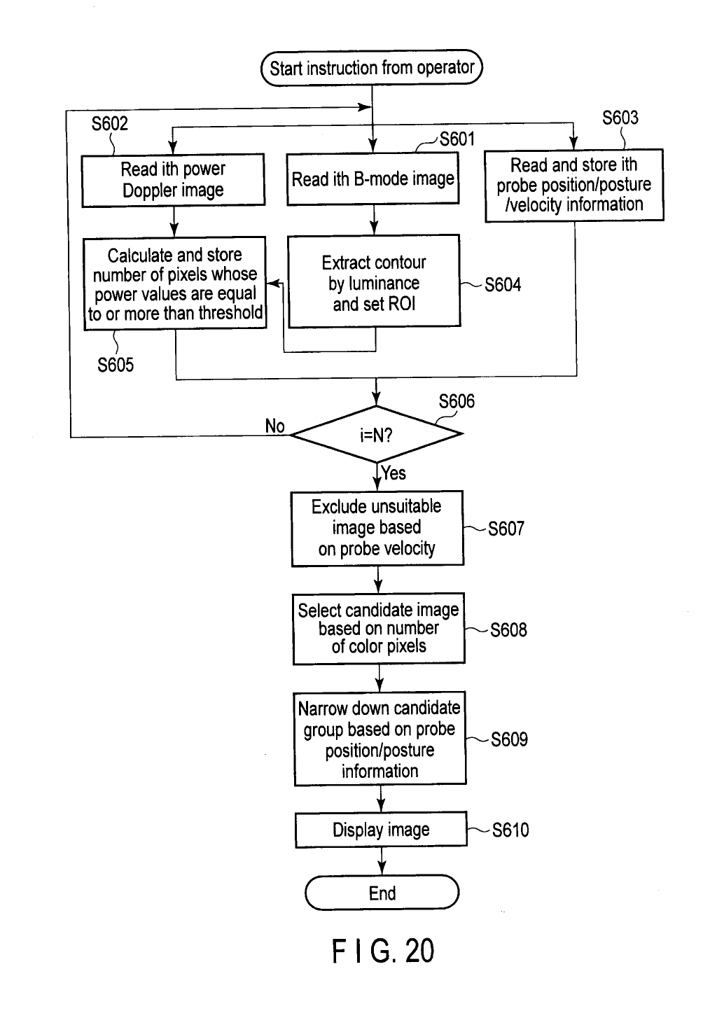

An operation procedure in this embodiment will be described with reference to the flowchart of FIG. 20. A basic operation procedure is the same as that in the third embodiment. However, this image processing apparatus differs from that in the third embodiment in that it reads B-mode image data and Doppler image data from a network connected to a data input/output unit 105 or an external storage device 106 and processes the data without performing ultrasonic transmission/reception or generating any image data.

Assume that in the following description, the ultrasonic diagnostic apparatus connected to a network has already executed the processing in steps S301 to S303 and S306 in the third embodiment, and has stored, in the external storage device 106, B-mode image data and Doppler image data corresponding to phases 1 to N (N is an integer), together with the position (x, y, z), posture (θx, θy, θz), and velocity (vx, vy, vz) of an ultrasonic probe 2, in a form that enables the discrimination of phases of image generation.

When the operator inputs an instruction to start processing by operating an operation panel 103, a control processor 101 reads B-mode image data corresponding to the ith phase from the external storage device 106 via the data input/output unit 105 and stores the read data in the storage unit 104 (step S601). The control processor 101 then reads Doppler image data corresponding to the ith phase from the external storage device 106 and stores the read data in the storage unit 104 (step S602). The control processor 101 also reads the position (x, y, z), posture (θx, θy, θz), and velocity (vx, vy, vz) corresponding to the ith phase from the external storage device 106 and stores them in a storage unit 104 (step S603).

Subsequently, the control processor 101 sets a region of interest (second region of interest) by using the same technique as that in step S105 based on the B-mode image data read in step S601 (step S604). In addition, the control processor 101 calculates the number of color pixels of a Doppler signal contained in the second region of interest set in step S604 in the Doppler image data read in step S602 by using the same technique as that in step S106 (step S605). The control processor 101 stores the calculated number of color pixels in the storage unit 104 in association with the Doppler image data stored in the storage unit 104 in step S602.

After step S605, the control processor 101 determines whether the counter i has reached N (step S606). If the counter i has not reached N (NO in step S606), the control processor 101 increments the counter i by one to execute steps S601 to S605 again.

When the counter i has reached N afterward (YES in step S606), the control processor 101 executes processing for selecting candidate image data suitable for diagnosis from the plurality of B-mode image data and the plurality of Doppler image data sequentially stored in repeatedly executed steps S601 and S602 (steps S607 to S609). The control processor 101 displays the selected candidate image data on the monitor 102 (step S610). Since steps S607 to S610 are the same as steps S308 to S311, a description of them will be omitted.

The image processing apparatus according to this embodiment can obtain the same effects as those of the third and fourth embodiments.

### (Modification)

The arrangement disclosed in each embodiment can be modified as needed.

For example, in each embodiment, it is possible to omit operations concerning several steps shown in the flowcharts of FIGS. 2, 7, 14, 18, and 19. In addition, the execution order of operations concerning the respective steps may be changed as needed.

In addition, it is possible to omit the operation of excluding image data unsuitable for diagnosis in accordance with average velocity values and the like (steps S108, S206, S308, S407, S505, and S607). In addition, it is possible to exclude image data suitable for diagnosis based on the numbers of color pixels in steps S108, S308, S407 and S607 as in steps S206 and S505.

In addition, it is possible to omit the operation of setting the second region of interest (steps S105, S304, S404, and S604). In this case, it is possible to use the first region of interest or a predetermined another region of interest as a region of interest for the calculation of the number of color pixels.

In each embodiment concerning the ultrasonic diagnostic apparatus, it is possible to perform setting of the second region of interest (steps S105 and S304) and calculation/storage of the number of color pixels (steps S106, S204, and S305) after the operator inputs an instruction to stop scanning.

In addition, it is possible to calculate image similarities based on Doppler image data instead of B-mode image data or both image data.

Furthermore, each embodiment has exemplified the case of using the number of color pixels in Doppler image data (especially power Doppler image data) as a parameter used for the selection of image data. Recently, when performing ultrasonic diagnosis of rheumatoid arthritis, it is a general practice to observe the inside of a joint cavity in the power Doppler mode and perform determination amount scoring by using the ratio of the number of color pixels occupying the joint cavity as a criterion. It is therefore conceivable to more effectively select image data useful for diagnosis based on the numbers of color pixels. However, a parameter used for the selection of image data in each embodiment need not always be the number of color pixels. For example, it is possible to use the total number of power values of color pixels as the parameter. This makes it possible to increase the robustness against the influence of a color pixel with a small power value such as a noise signal and preferentially select image data clearly containing signals with high blood flow densities.

Furthermore, it is possible to use the sum total of velocity values of color pixels as the above parameter. This parameter is useful when the operator wants to preferentially extract an image containing a blood flow with a high blood flow rate.

In addition, each embodiment has exemplified the case of displaying selected image data on the monitors 4 and 102. However, it is possible to add a tag to selected image data to identify it against other image data so as to easily allow browsing of the image data afterward, instead of directly displaying the image data on the monitors 4 and 102. In addition, when referring to image data acquired in the past while continuously switching over them with the operation of a trackball or the like, it is possible to stop switching over at the position of the image to which the tag is added.

Furthermore, in the third and sixth embodiments, steps S310 and S609 have exemplified the case of selecting image data by using both the position (x, y, z) and posture (θx, θy, θz) of the ultrasonic probe 2. However, consideration may be given to only one of them. In addition, it is not necessary to use all triaxial values concerning the position (x, y, z), posture (θx, θy, θz), and velocity (vx, vy, vz) in steps S308, S310, S607, and S609.

Some embodiments of the present invention have been described above. However, these embodiments are presented merely as examples and are not intended to restrict the scope of the invention. These novel embodiments can be carried out in various other forms, and various omissions, replacements, and alterations can be made without departing from the spirit of the invention. The embodiments and their modifications are also incorporated in the scope and the spirit of the invention as well as in the invention described in the claims and their equivalents.

1. An ultrasonic diagnostic apparatus comprising:
a transmitter/receiver which repeats ultrasonic transmission/reception with respect to a subject;
an image generator which generates data of a plurality of images based on an output from the transmitter/receiver;
a blood flow image generator which generates data of a plurality of blood flow images based on an output from the transmitter/receiver;
a similarity calculator which calculates similarities between the plurality of images;
a specifying processor which specifies at least two images exhibiting a low similarity from the plurality of images based on the similarities;
an image selector which selects at least two blood flow images respectively corresponding to scanning times of the specified at least two images from the plurality of blood flow images; and
a display which displays the selected at least two blood flow images.

2. The ultrasonic diagnostic apparatus according to claim 1, wherein the image comprises a morphological image.

3. The ultrasonic diagnostic apparatus according to claim 1, wherein the image comprises a Doppler image, a contrast-enhanced blood vessel image, or an elastography image.

4. The ultrasonic diagnostic apparatus according to claim 1, wherein the blood flow image comprises a Doppler image or a contrast-enhanced blood vessel image.

5. The ultrasonic diagnostic apparatus according to claim 1, wherein the similarity calculator calculates a similarity between a reference image selected from the plurality of images and each of the remaining images.

6. The ultrasonic diagnostic apparatus according to claim 5, wherein the similarity calculator selects, as the reference image, an image generated first among the plurality of images.

7. The ultrasonic diagnostic apparatus according to claim 5, wherein the similarity calculator selects, as the reference image, an image corresponding to a blood flow image, of the plurality of blood flow images, whose number of blood flow pixels has reached a threshold first, from the plurality of images.

8. The ultrasonic diagnostic apparatus according to claim 1, further comprising a blood flow image exclusion processor which excludes a blood flow image, of the plurality of blood flow images, which exhibits a pixel value not less than a threshold from display targets.

9. The ultrasonic diagnostic apparatus according to claim 1, wherein the display superimposes and displays a blood flow image selected by the image selector on a morphological image of the subject and also displays a blood flow area or an area ratio in a predetermined region in the blood flow image.

10. The ultrasonic diagnostic apparatus according to claim 8, wherein the blood flow image expresses, in color pixel, a position where a blood flow is observed, and
the blood flow image exclusion processor calculates, as the number of blood flow pixels, the number of color pixels contained in a predetermined region of interest in each of the plurality of blood flow images.

11. The ultrasonic diagnostic apparatus according to claim 1, wherein the similarity calculator calculates a mean square error between the plurality of images as the similarity.

12. The ultrasonic diagnostic apparatus according to claim 1, wherein the blood flow image expresses, in color pixel, a position where a blood flow is observed, and
the similarity calculator calculates the similarity based on a temporal change in the number of color pixels contained in a predetermined region of interest in each of the plurality of blood flow images.

13. The ultrasonic diagnostic apparatus according to claim 12, wherein the similarity calculator segments a series of the plurality of images and the plurality of blood flow images into a plurality of image groups based on a magnitude of the temporal change, and calculates the image similarities such that similarities between image groups belonging to the respective segments become high.

14. The ultrasonic diagnostic apparatus according to claim 1, further comprising an excluding processor which excludes a blood flow image, of the plurality of images and the plurality of blood flow images, whose average blood flow velocity value or variance value is larger than a predetermined threshold and an image corresponding to the blood flow image from targets of selection by the image selector.

15. The ultrasonic diagnostic apparatus according to claim 1, further comprising:
a tissue velocity calculator which calculates an average tissue velocity value or a variance value in a predetermined region of interest when the image generator and the blood flow image generator generates the plurality of images and the plurality of blood flow images; and
an excluding processor which excludes a blood flow image whose average tissue velocity or variance value is larger than a predetermined threshold and an image corresponding to the blood flow image from targets of selection by the image selection unit.

16. An ultrasonic diagnostic apparatus comprising:
an ultrasonic probe;
a transmitter/receiver which repeats ultrasonic scanning on a subject through the ultrasonic probe;
a detector which detects a position of the ultrasonic probe;
a blood flow image generator which generates data of a plurality of blood flow images based on an output from the transmitter/receiver;
an image selector which selects at least two blood flow images having a low similarity from the plurality of blood flow images based on a position of the ultrasonic probe; and
a display which displays the selected at least two blood flow images.

17. The ultrasonic diagnostic apparatus according to claim 16, wherein the image selector calculates a reciprocal of a moving distance of the ultrasonic probe as an index indicating a similarity.

18. The ultrasonic diagnostic apparatus according to claim 16, further comprising a blood flow image exclusion processor which excludes a blood flow image, of the plurality of blood flow images, which exhibits a pixel value not less than a threshold from display targets.

19. The ultrasonic diagnostic apparatus according to claim 16, further comprising an excluding processor which excludes a blood flow image which exhibits a change in position detected by the detector when the blood flow image generator generates the plurality of images and the plurality of blood flow images exceeds a threshold from targets of selection by the image selector.

20. An image processing apparatus comprising:
a storage which stores data of a plurality of ultrasonic images and data of a plurality of blood flow images concerning a subject;
a similarity calculator which calculates similarities between the plurality of ultrasonic images;
a specifying processor which specifies at least two ultrasonic images exhibiting a low similarity from the plurality of ultrasonic images based on the similarities;
an image selector which selects at least two blood flow images respectively corresponding to scanning times of the specified at least two ultrasonic images from the plurality of blood flow images; and
a display which displays the selected at least two blood flow images.

21. An image processing method comprising:
calculating similarities between the plurality of images in data of a plurality of ultrasonic images and data of a plurality of blood flow images concerning a subject;
specifying at least two images exhibiting a low similarity from the plurality of images based on the similarities;
selecting at least two blood flow images respectively corresponding to scanning times of the specified at least two images from the plurality of blood flow images; and
displaying the selected at least two blood flow images.
